(19) United States
(12) Patent Application Publication
(10) Pub. No.: US 2022/0135702 A1
(43) Pub. Date: May 5, 2022

(54) NEUROPILIN ANTIBODY NASAL SPRAY TO REDUCE COVID-19 TRANSMISSION

(71) Applicant: MARV ENTERPRISES LLC, Hermitage, PA (US)

(72) Inventor: Mitchell S. Felder, Hermitage, PA (US)

(21) Appl. No.: 17/514,898

(22) Filed: Oct. 29, 2021

Related U.S. Application Data
(60) Provisional application No. 63/108,301, filed on Oct. 31, 2020.

(51) Int. Cl. C07K 16/40; C07K 16/28; C07K 16/10; A61K 9/00; A61K 47/44; A61K 47/02

(52) U.S. Cl. CPC C07K 16/40; C07K 16/2863; A61K 47/02; A61K 9/0043; A61K 47/44; C07K 16/10

(57) ABSTRACT

An embodiment provides a method for preventing the transmission of Covid-19, including: preparing a composition comprising an ACE2 monoclonal antibody, a Neuropilin-1 antibody, an antibody to the spike protein of Sars-CoV-2, a vegetable oil, and a saline solution; and spraying the composition in a nasal cavity of a patient. Other aspects are described and claimed.

### CROSS REFERENCE TO RELATED APPLICATION

This application claims priority to U.S. Provisional Patent Application Ser. No. 63/108,301, filed on Oct. 31, 2020, and entitled “NASAL SPRAY TO PREVENT THE TRANSMISSION OF THE COVID-19 VIRUS,” the contents of which are incorporated by reference herein.

### FIELD

This application relates generally to a method for the reduction of transmission of COVID-19, and, more particularly, to a nasal spray to reduce COVID-19 transmission.

### BACKGROUND

Coronaviruses are a family of viruses that can cause illnesses such as severe acute respiratory syndrome (SARS) and Middle East respiratory syndrome (MERS). A new coronavirus (Covid-19) was identified as the cause of a disease outbreak in China. The virus is presently known as the severe acute respiratory syndrome coronavirus 2 (SARS-CoV-2). The disease it causes is called coronavirus disease 2019 (COVID-19).

Cases of COVID-19 have been reported in multiple countries, where it has caused a great deal of morbidity and mortality, in a worldwide pandemic. The disorder is characterized by shortness of breath, increased mucus production, sore throat, cough, and fever. This may necessitate admission to a hospital, with subsequent admission to an intensive care unit for the respiratory support of the infected patient.

There is a need for the prevention or reduction in spread of COVID-19, due to the worldwide pandemic of this infection.

### BRIEF SUMMARY

In summary, one embodiment provides a composition for preventing the transmission of Covid-19, comprising: an ACE2 monoclonal antibody; a Neuropilin-1 antibody; an antibody to the spike protein of Sars-CoV-2; a vegetable oil; and a saline solution, wherein the ACE2 monoclonal antibody, the Neuropilin-1 antibody, the antibody to the spike protein of Sars-CoV-2, the vegetable oil, and saline solution are combined into a nasal sprayable version of the composition.

Another embodiment provides a method for preventing the transmission of Covid-19, comprising: preparing a composition comprising an ACE2 monoclonal antibody, a Neuropilin-1 antibody, an antibody to the spike protein of Sars-CoV-2, a vegetable oil, and a saline solution; and spraying the composition in a nasal cavity of a patient.

A further embodiment provides a kit for preventing the transmission Covid-19, comprising: a composition comprising, an ACE2 monoclonal antibody capable of blocking ACE2 receptors of a patient, a Neuropilin-1 antibody, an antibody to the spike protein of Sars-CoV-2, a sesame oil, and a saline solution; and a sprayer for placing the composition in a nasal cavity of the patient.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example flow diagram of a method of a nasal spray for reducing Covid-19 transmission.

### DETAILED DESCRIPTION

It will be readily understood that the components of the embodiments, as generally described and illustrated in the FIGURES herein, may be arranged and designed in a wide variety of different configurations in addition to the described example embodiments. Thus, the following more detailed description of the example embodiments, as represented in the FIGURES, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

Covid-19 has spread worldwide and become a global pandemic. The loss of life, suffering, and economic struggles have reached all corners of the globe. Symptoms may manifest about 2-14 days after exposure. The symptoms may include fever, chills, cough, shortness of breath, difficulty breathing, fatigue, muscle/body aches, new loss of taste/smell, sore throat, congestion, runny nose, nausea, vomiting, or diarrhea. More severe symptoms may include trouble breathing, persistent pain/pressure in the chest, confusion, inability to wake or stay awake, or bluish lips/face. Some cases may require hospitalization and even intensive care unit healthcare. Because of the novelty of the virus, very few tests exist that are specific for Covid-19. What is needed is a method for the reduction of Covid-19 transmission.

Coronaviruses are a family of viruses that can cause illnesses such as severe acute respiratory syndrome (SARS) and Middle East respiratory syndrome (MERS). A new coronavirus (Covid-19) was identified as the cause of a disease outbreak in China. The virus is known as the severe acute respiratory syndrome coronavirus 2 (SARS-CoV-2). The disease it causes is called coronavirus disease 2019 (Covid-19).

Angiotensin-Converting Enzyme 2 (ACE2) is the main receptor for SARS-CoV-2. In the human lung, ACE2 is expressed on type I and II alveolar epithelial cells. Men have shown a higher ACE2 level in their alveolar cells than women. ACE2 is a member of the Angiotensin Converting Enzyme (ACE) family of dipeptidyl-carboxy-dipeptidase, is highly homologous to ACE1. ACE2 has a high affinity to Ang II type 1 and type 2 receptors and plays an important role in cell proliferation and hypertrophy, inflammatory response, blood pressure, and fluid balance. ACE2 is highly expressed in certain organs and tissues. In a phylogenetic analysis of 103 strains of SARS-CoV-2 from China, two different types of SARS-CoV-2 were identified, designated type L (accounting for 70 percent of the strains) and type S (accounting for 30 percent). The strains in L type, derived from S type, are evolutionarily more aggressive and contagious.

SARS-CoV-2 may bind to ACE2 expressed on the nasal mucosal epithelial cell, and after about 96 hours enters human lower respiratory epithelial cells. ACE2 is abundantly expressed in the lungs. The expression of ACE2 has been found to be much less in children, which may be a reason for their lower infectious rate. ACE2 increases in abundance as a child grows, which is a reason that less than two percent of all individuals infected with SARS-CoV-2 are children.

Neuropilin-1 is a protein that in humans is encoded by the NRP1 gene. Neuropilin-1 is a coreceptor to a tyrosine kinase receptor for semaphoring and vascular endothelial growth factor. This protein is important in cell survival, migration, and angiogenesis. Neuropilin-1 facilitates the entry of SARS-CoV-2 into nasal epithelial cells, and subsequently into the bloodstream of the infected individual. Neuropilin-1 is known to bind furin-cleaved substrates, and therefore significantly potentiates SARS-CoV-2 infectivity. This may be blocked by utilizing a monoclonal blocking antibody against Neuropilin-1. Neuropilin-1 helps to allow for the cellular entry of SARS-CoV-2, into a host via the nasal epithelium, along with ACE2 and the Spike Protein of SARS-CoV-2. It is also possible that SARS-CoV-2 can enter cells independently of ACE2 when viral loads are high. Blocking Neuropilin-1, ACE2 and the Spike Protein of SARS-CoV-2 greatly reduces the viral load entering the host. This leads to a greatly reduced viral inoculum, in each infected individual, with a concomitant reduction in clinical morbidity and mortality. This could also potentially lead to the rapid herd immunity of a given population.

Based on the unique anatomical structure of olfactory nerves and olfactory bulb, the transmission may become a channel between the nasal epithelium and the central nervous system (CNS). Once latched onto the nasal respiratory cell receptors the SARS-CoV2 may engineer its fusion with the cell membranes. As an intracellular mechanism the viral RNA may be released intracellularly, followed by its translation and formation of a replication complex that produce more viral particles, which are then packaged in the Golgi apparatus of the cell and finally released to the intracellular space to renew its attack on other cells. SARS-CoV-2 has been recovered from nasal secretions, feces, and blood, after it has entered the blood stream via the olfactory nerve.

The clinical symptoms of SARS-CoV-2 infection vary a great deal from patient to patient but include shortness of breath, increased mucus production, sore throat, cough, and fever. The virus first affects the respiratory epithelial cells and alveolar cells, followed by the digestive system. This may necessitate admission to a hospital, with subsequent admission to an intensive care unit for the respiratory support of the infected patient. Viral infections can produce, in some, an excessive immune reaction in the host labeled a Cytokine Storm, that causes an acute systemic inflammatory syndrome characterized by fever and multiple organ dysfunction. There is therefore a need for treatments to reduce Covid-19 transmission between humans. The aim of this invention is to prevent the initial transmission into the nasal mucosal epithelial cells, thereby preventing the entire sequence of pathology.

Accordingly, described herein is a method and composition for the prevention of transmission of Covid-19. In an embodiment, the composition may be a nasal spray placed in the nasal cavity of a human. The spray may require reapplication. The composition may comprise Neuropilin-1 antibodies. The composition may comprise SARS-CoV-2 spike protein antibodies. The composition may comprise an ACE2 antibody. The antibody or antibodies may be monoclonal. The antibody may bind ACE2 receptors in the nasal cavity and/or elsewhere in the respiratory tract. The binding of ACE2 receptors may prevent the mechanism of uptake of the Covid-19 virus in a cell and its replication. The composition may comprise a vegetable oil such as coconut, olive, sesame, or the like, or a combination thereof. The composition may comprise saline. The osmolarity of the composition may be adjusted to the physiological osmolarity of a patient.

The illustrated example embodiments will be best understood by reference to the FIGURES. The following description is intended only by way of example, and simply illustrates certain example embodiments.

In an embodiment, a composition of matter and a method using the composition may be used that reduces the transmission of Covid-19 between humans. The composition includes a nasal spray comprising at least one vegetable oil, saline solution, and sufficient ACE2 antibody, Neuropilin-1 antibody, and SARS-CoV-2 spike protein antibody for efficacy. In an embodiment, the administering of the nasal spray composition may use a standard nasal spray bottle. The composition when administered using the method may be effective in reducing the transmission of Covid-19 for an extended period.

In an embodiment, a composition comprises an ACE2 monoclonal antibody, Neuropilin-1 antibody, and SARS-CoV-2 spike protein antibody in a fluid. The composition may be sprayed into the nose and may block ACE2 receptors, Neuropilin-1 protein, and impede the replication of SARS-CoV-2. The blocking may occur for at least one hour and up to about a week. The fluid may include at least one vegetable oil and/or a saline solution. In an embodiment, the volumetric ratios of vegetable oil and saline solution in the fluid range from 0.1 to 10. ACE2 antibody, Neuropilin-1 antibody, and SARS-CoV-2 spike protein antibody may be added to the fluid in a sufficient amount for efficacy. Preferably, the composition should be refrigerated at a temperature of 1-10° C. prior to spraying into each nostril of the nose. The method may comprise spraying the composition into each nostril and releasing at least about 0.05 mL of the composition into each nostril.

The vegetable oil may include a hydrophobic element. The vegetable oil may be selected from a group consisting of coconut oil, olive oil, sesame oil, and combinations thereof. Most preferably, the vegetable oil consists essentially of sesame oil.

The saline solution comprises a solution of sodium chloride and water. The saline solution may have an osmolarity similar in osmolarity to bodily tissue. The saline solution may comprise a normal saline solution, which is a mixture of salt and water (0.9% NaCl) and is an isotonic fluid containing water, sodium (154 mEq/L) and chloride (154 mEq/L) similar in concentration to bodily fluids.

The ACE2 antibody can include a commercially produced monoclonal ACE2 antibody, such as, for example, Proteintech (Proteintech, Rosemont, Ill., USA: Cat No. 66699-1-Ig). Other commercial antibodies may be used. An alternative ACE2 antibody may also be created utilizing standard laboratory procedures well known in the art.

In embodiments, the composition includes 10-25 ml of sesame oil, at least 5 ml of saline solution, and a combination of at least 150 μl of ACE2 antibody, 150 μl Neuropilin-1 antibody, and 150 μl antibody to the Spike Protein of Sars-CoV-2. In one embodiment, the composition comprises 10 mL of sesame oil, 10 mL normal saline solution, and 150 μl of Proteintech or other ACE2 antibody, 150 μl Neuropilin-1 antibody, and 150 μl antibody to the Spike Protein of Sars-CoV-2. In an alternative embodiment, the composition includes 20 ml of a fluid comprising a 1:1 sesame oil:normal saline solution with up to 2000 μl of laboratory produced monoclonal ACE2 antibody, Neuropilin-1 antibody, and an antibody to the Spike Protein of Sars-CoV-2.

In an embodiment, the composition may be created with a longer shelf life. A variant of the composition, for prolongation of its efficacy, utilizes monomers which form a biodegradable polymer matrix for the slow release of the ACE2 monoclonal antibodies into the nose. Slow-release drug delivery technologies are well known to one skilled in the art and include commercially available products such as SynBiosys® (InnoCore Pharmaceuticals, Groningen, The Netherlands, a registered trademark in the US and other countries). The technology uses several monomers to form a biodegradable polymer matrix in which monoclonal antibodies against ACE2, Neuropilin-1 antibodies, and antibodies to the Spike Protein of Sars-CoV-2 can be entrapped. When brought into an aqueous environment, the polymers swell and gradually release the monoclonal antibodies by diffusion. Release time can be regulated from hours up to days. The SynBiosys® polymers may comprise DL-lactide, glycolide, ε-caprolactone and polyethyleneglycol-biologically monomers. A SynBiosys® feature is that it allows sustained release of the composition over time.

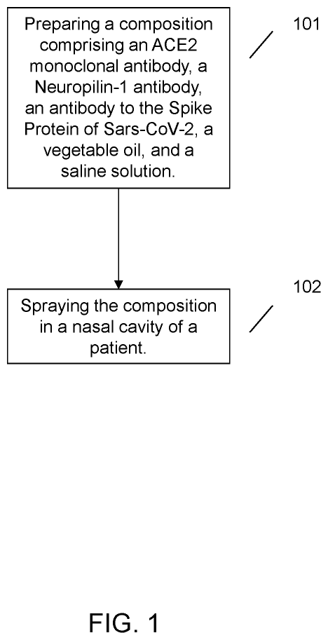

Referring to FIG. 1, an example method is illustrated. In an embodiment, a method for preventing the transmission of Covid-19 may include preparing a composition comprising an ACE2 monoclonal antibody, Neuropilin-1 antibodies, antibodies to the Spike Protein of Sars-CoV-2, a vegetable oil, and a saline solution at 101. In an embodiment, the composition may be sprayed in a nasal cavity of a patient at 102.

In an embodiment, the method may include an assay for determining the efficacy or a concentration of an antibody in a patient receiving the nasal spray. For example, the assay may determine an antibody level, a binding to a target antigen, or the like. The assay may be automated. For example, a body fluid may be drawn, analyzed, and an output provided to determine antibody efficacy. Such an assay may be used to determine a timing or dosage of a nasal spray application is required or altered.

An exemplar embodiment has been described above. However, it will be apparent to those skilled in the art that numerous variations of the type described could be made to the present invention without departing from the spirit of the invention. The scope of the present invention is defined by the broad general meaning of the terms in which the claims are expressed.

Unless otherwise indicated, all numbers expressing quantities of ingredients, properties such as molecular weight, reaction conditions, and so forth used in the specification and claims are to be understood as being modified in all instances by the term “about.”

Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

While the invention has been particularly shown and described with reference to a preferred embodiment, it will be understood by those skilled in the art that various changes in form and detail may be made therein without departing from the spirit and scope of the invention. All documents, books, manuals, papers, patents, published patent applications, guides, abstracts, and other references cited herein are incorporated by reference in their entirety.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data.

Embodiments may be implemented as an instrument, system, method or program product. Accordingly, an embodiment may take the form of an entirely hardware embodiment, or an embodiment including software (including firmware, resident software, micro-code, etc.) that may all generally be referred to herein as a “circuit,” “module” or “system.” Furthermore, embodiments may take the form of a program product embodied in at least one device readable medium having device readable program code embodied thereon.

A combination of device readable storage medium(s) may be utilized. In the context of this document, a device readable storage medium (“storage medium”) may be any tangible, non-signal medium that can contain or store a program comprised of program code configured for use by or in connection with an instruction execution system, apparatus, or device. For the purpose of this disclosure, a storage medium or device is to be construed as non-transitory, i.e., not inclusive of signals or propagating media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the FIGURES, which illustrate example methods, devices and products according to various example embodiments. It will be understood that the actions and functionality may be implemented at least in part by program instructions. These program instructions may be provided to a processor of a device, e.g., a hand-held measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying FIGURES, it is to be understood that this description is not limiting and that various other changes and modifications may be affected therein by one skilled in the art without departing from the scope or spirit of the disclosure.

What is claimed is:

1. A composition for preventing the transmission of Covid-19, comprising:
an ACE2 monoclonal antibody;
a Neuropilin-1 antibody;
an antibody to the spike protein of Sars-CoV-2;
a vegetable oil; and
a saline solution, wherein the ACE2 monoclonal antibody, the Neuropilin-1 antibody, the antibody to the spike protein of Sars-CoV-2, the vegetable oil, and saline solution are combined into a nasal sprayable version of the composition.

2. The composition of claim 1, wherein the vegetable oil is selected from the group consisting of coconut oil, olive oil, and sesame oil.

3. The composition of claim 1, further comprising storing the composition at a temperature of 1-10 degrees Celsius.

4. The composition of claim 1, further comprising adding at least one monomer to form a biodegradable polymer matrix.

5. The composition of claim 1, wherein the saline adjusts the osmolarity of the composition to be similar to the osmolarity of a patient.

6. The composition of claim 1, wherein the saline comprises 0.9% sodium chloride.

7. The composition of claim 1, wherein a volumetric ratio of the vegetable oil and the saline solution range from 0.1 to 10.

8. The composition of claim 1, wherein a concentration of the ACE2 monoclonal antibody, the Neuropilin-1 antibody, and the antibody to the spike protein of Sars-CoV-2 are adjusted for a binding efficiency.

9. The composition of claim 1, wherein the Neuropilin-1 antibody blocks cellular entry of SARS-CoV-2 into a host via the nasal epithelium.

10. The composition of claim 1, further comprising spraying the composition into a nasal cavity of a patient.

11. A method for preventing the transmission of Covid-19, comprising:
preparing a composition comprising an ACE2 monoclonal antibody, a Neuropilin-1 antibody, an antibody to the spike protein of Sars-CoV-2, a vegetable oil, and a saline solution; and
spraying the composition in a nasal cavity of a patient.

12. The method of claim 11, wherein the vegetable oil is selected from the group consisting of coconut oil, olive oil, and sesame oil.

13. The method of claim 11, further comprising storing the composition at a temperature of 1-10 degrees Celsius.

14. The method of claim 11, further comprising adding at least one monomer to form a biodegradable polymer matrix.

15. The method of claim 11, wherein the saline adjusts the osmolarity of the composition to be similar to the osmolarity of a patient.

16. The method of claim 11, wherein the saline comprises 0.9% sodium chloride.

17. The method of claim 11, wherein a volumetric ratio of the vegetable oil and the saline solution range from 0.1 to 10.

18. The method of claim 11, wherein a concentration of the ACE2 monoclonal antibody, the Neuropilin-1 antibody, and the antibody to the spike protein of Sars-CoV-2 are adjusted for a binding efficiency.

19. The method of claim 11, wherein the Neuropilin-1 antibody blocks cellular entry of SARS-CoV-2 into a host via the nasal epithelium.

20. A kit for preventing the transmission Covid-19, comprising:
a composition comprising, an ACE2 monoclonal antibody capable of blocking ACE2 receptors of a patient, a Neuropilin-1 antibody, an antibody to the spike protein of Sars-CoV-2, a sesame oil, and a saline solution; and
a sprayer for placing the composition in a nasal cavity of the patient.
